(19) United States
(12) Patent Application Publication
(10) Pub. No.: US 2024/0118107 A1
(43) Pub. Date: Apr. 11, 2024

(54) DYNAMIC SIGNAL TUNING FOR ACTIVE NOISE CANCELLATION

(71) Applicant: Meta Platforms Technologies, LLC, Menlo Park, CA (US)

(72) Inventors: Matthew Sherwood, Seattle, WA (US); Filipp Demenschonok, Bothell, WA (US)

(21) Appl. No.: 18/484,019

(22) Filed: Oct. 10, 2023

Related U.S. Application Data
(60) Provisional application No. 63/414,868, filed on Oct. 10, 2022.

(51) Int. Cl. G01D 3/036; G06F 3/01

(52) U.S. Cl. CPC G01D 3/036; G06F 3/015

(57) ABSTRACT

The disclosed method may include driving, using a biosensing device, a right-leg drive (RLD) signal, and measuring, using the biosensing device, a biosignal. The method also includes determining, from the measured biosignal, noise within the biosignal, and tuning, based on the noise, the RLD signal. Various other methods, systems, and computer-readable media are also disclosed.

### CROSS REFERENCE TO RELATED APPLICATION

This application claims the benefit of U.S. Provisional Application No. 63/414,868, filed 10 Oct. 2022, the disclosure of which is incorporated, in its entirety, by this reference.

### BRIEF DESCRIPTION OF THE DRAWINGS

The accompanying drawings illustrate a number of exemplary embodiments and are a part of the specification. Together with the following description, these drawings demonstrate and explain various principles of the present disclosure.

FIG. 1 is a simplified diagram of an exemplary circuit for dynamic signal tuning for active noise cancellation in a right-leg drive (RLD) system.

FIGS. 2A, 2B and 2C are simplified diagrams of example tuning circuits for dynamic signal tuning.

FIG. 3 is a flow diagram of an exemplary method for dynamic signal tuning for active noise cancellation.

FIG. 4 is an illustration of exemplary augmented-reality glasses that may be used in connection with embodiments of this disclosure.

FIG. 5 is an illustration of an exemplary virtual-reality headset that may be used in connection with embodiments of this disclosure.

FIGS. 6A and 6B are illustrations of an exemplary human-machine interface configured to be worn around a user's lower arm or wrist.

FIGS. 7A and 7B are illustrations of an exemplary schematic diagram with internal components of a wearable system.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the present disclosure covers all modifications, equivalents, and alternatives falling within the scope of the appended claims.

### DETAILED DESCRIPTION OF EXEMPLARY EMBODIMENTS

Biosignal measurements involve measuring electrical signals from the human body using biosensing devices such as electrocardiograms (ECG), electroencephalograms (EEG), and electromyographs (EMG), etc. These electrical signals may be used to measure certain aspects of the body, such as in a medical diagnostic context. These electrical signals may also be used as inputs for input devices of computing devices.

The human body itself may act as a conductor capable of receiving electromagnetic interference (EMI) from nearby electrical signals, akin to an antenna. One such signal may include a common-mode signal. A common-mode signal may be a component of an analog signal having the same or common sign (e.g., + or −) that may be present on all conductors of a circuit. The common-mode signal may be induced by electromagnetic fields, which may be generated from oscillating voltages (e.g., changes in current), from nearby electrical sources. However, when measuring biosignals, the human body may exhibit an undesirable common-mode signal induced from nearby electronic devices, motors, electrical power lines (e.g., low frequency power line interference (PLI)), radio signals, etc. This common-mode signal present in the human body may act as interference, which may be known as common-mode interference, that may reduce the accuracy of the biosignal measurements by introducing noise. Other noise sources, which may be internal and/or external, may include radiation and conducted coupling mechanisms. Due to the precision necessary for accurate measurements, any noise may greatly influence accuracy.

Normally, the human body may be floating electrically, for instance because the human body may not normally be grounded to a particular voltage. As such, the human body may be susceptible to common-mode interference. An electrical circuit may be used to reduce or cancel this common-mode interference by actively driving an electrical signal—which may be a known voltage (e.g., a ground voltage)—into the body. Traditionally, the electrical circuit may drive the known voltage into the body part furthest from the heart (e.g., the right leg). Named for the right leg, right leg drive (RLD) circuits may reduce common-mode interference by applying an electrode to the right leg for driving the known voltage. Thus, biosensing devices may include RLD circuits. However, biosensing devices often experience variabilities, such as electrode movement or liftoff, changes in noise sources, etc., that may affect an effectiveness of an RLD signal. Conventional RLD circuits may be unable to adapt to variabilities.

The present disclosure is generally directed to dynamic signal tuning for active noise cancellation. As will be explained in greater detail below, embodiments of the present disclosure may measure, in response to driving an RLD signal, a biosignal and determine a noise within the measured biosignal to tune the RLD signal. By continuously measuring and analyzing biosignals for noise and dynamically tuning the RLD signal based on the noise, the systems and methods described herein may more effectively cancel noise using the RLD signal.

Features from any of the embodiments described herein may be used in combination with one another in accordance with the general principles described herein. These and other embodiments, features, and advantages will be more fully understood upon reading the following detailed description in conjunction with the accompanying drawings and claims.

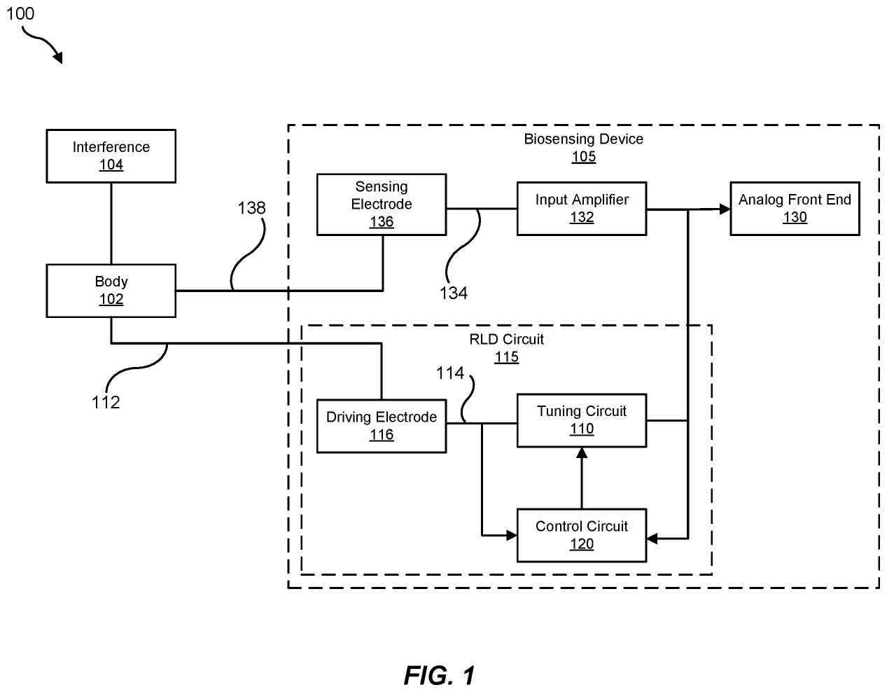

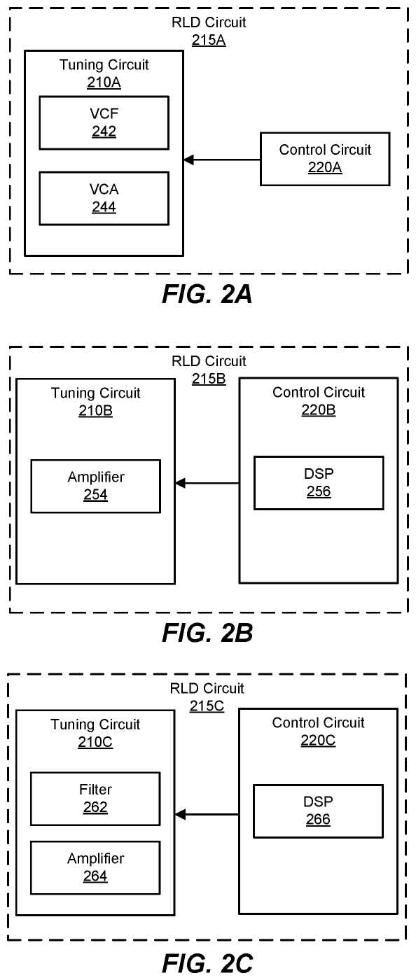

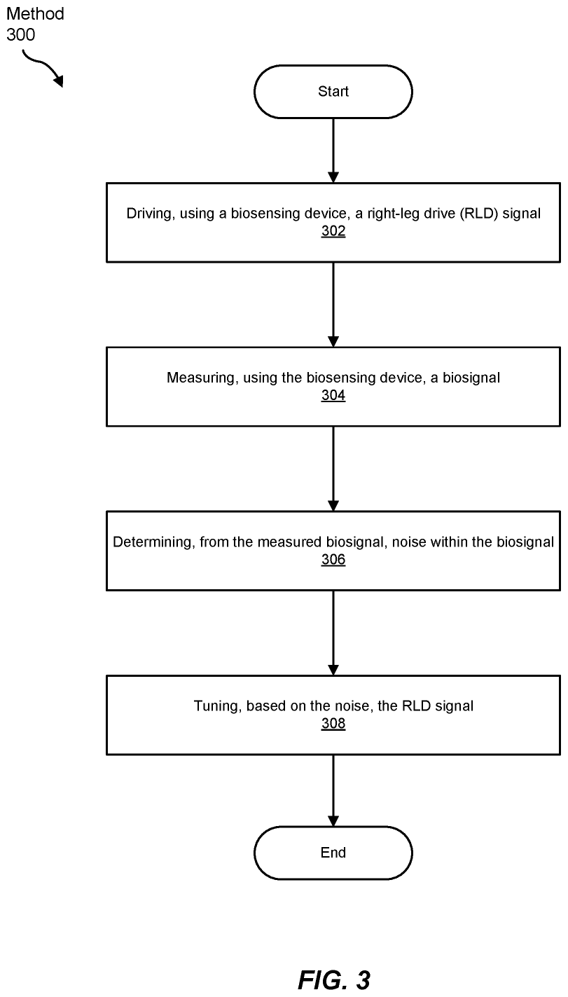

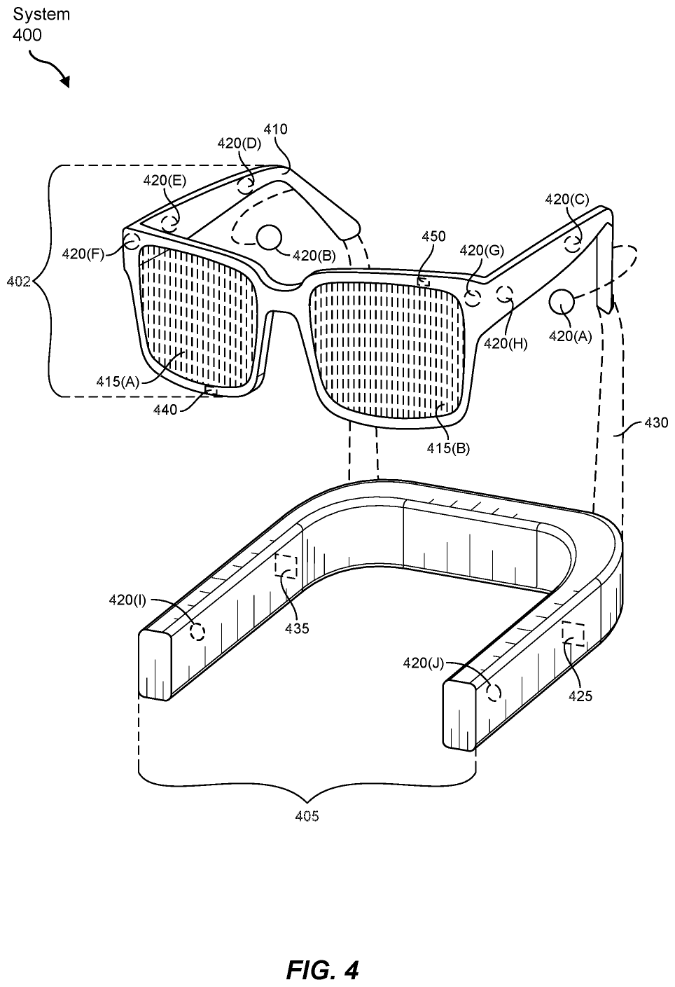

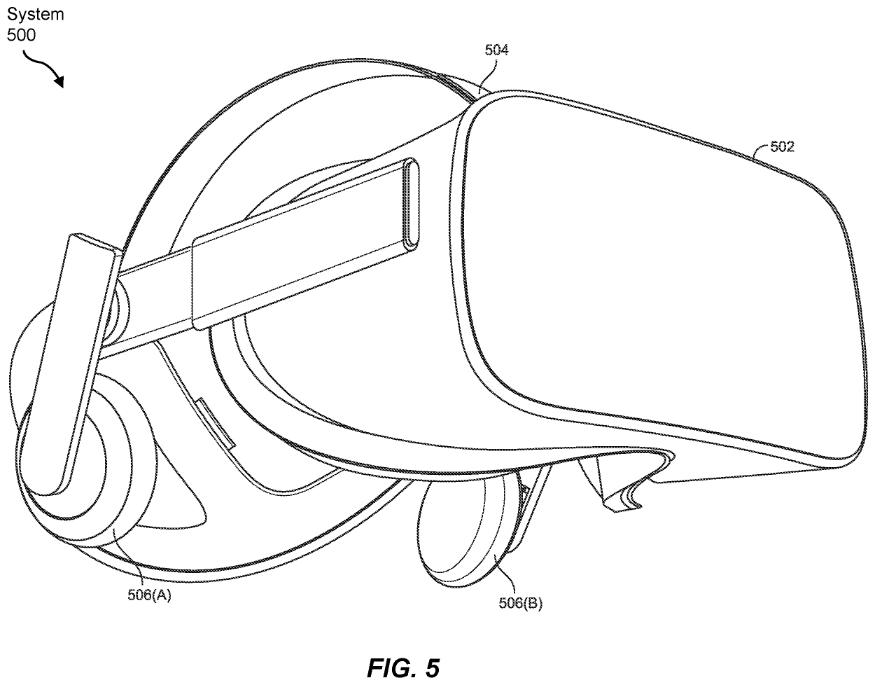

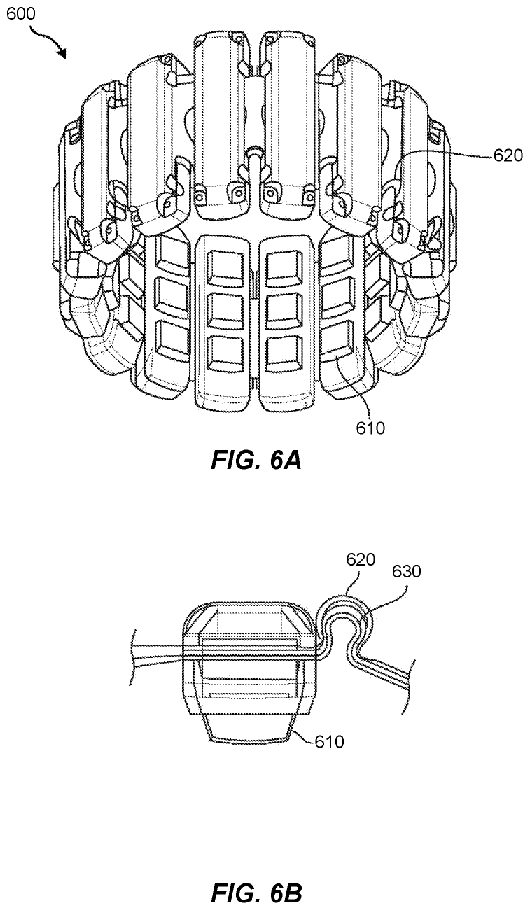

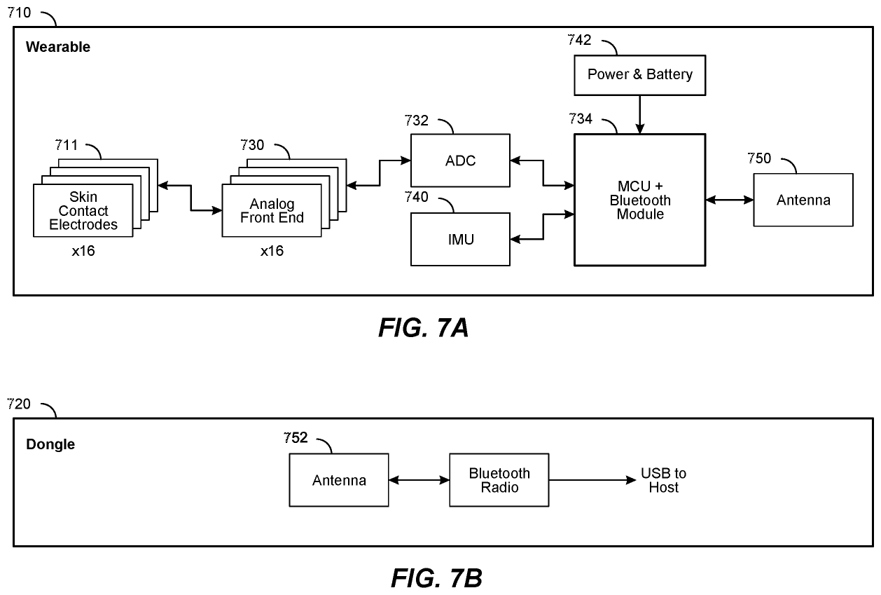

The following will provide, with reference to FIGS. 1-7B, detailed descriptions of dynamic tuning for active noise cancellation. Detailed descriptions of an example biosensing device will be provided in connection with FIG. 1. Detailed descriptions of example tuning circuits for dynamic tuning of a noise cancelling signal will be provided in connection with FIGS. 2A-2C. Detailed descriptions of an example method for dynamically tuning a noise cancelling signal will be provided in connection with FIG. 3. Detailed descriptions of example headset devices will be provided in connection with FIGS. 4 and 5. Detailed descriptions of example wearable devices will also be provided in connection with FIGS. 6A-7B.

FIG. 1 illustrates a biosensing environment 100 of a biosensing device 105 worn by a body 102 coupled to an interference 104. Biosensing device 105 may include a sensing electrode 136, an effective impedance 134, an input amplifier 132, an analog front end (AFE) 130, and RLD circuit 115, a driving electrode 116, a feedback impedance 114, a tuning circuit 110, and a control circuit 120. FIG. 1 also illustrates a biosignal 138 and an RLD signal 112.

As described herein, body 102 may receive interference 104, which may correspond to PLI (e.g., 50/60 Hz), narrowband interference, and/or other EMI. Biosensing device 105 may measure biosignal 138 from body 102 using sensing electrode 136. Sensing electrode 136 may correspond to one or more electrodes directly contacting body 102 (e.g., a skin of body 102) for measuring biosignal 138. Sensing electrode 136 may exhibit effective impedance 134 that in some examples may be inversely proportional to a gain of a signal fed back into body 102. Input amplifier 132, which in some examples may be part of analog front end 130, may amplify biosignal 138 for further processing by AFE 130 (e.g., as biological measurements, user inputs, etc.) which may include a microprocessor, controller, and/or other processor.

RLD circuit 115 may include driving electrode 116 that may drive RLD signal 112 into body 102. Although described herein as an RLD signal, in other examples RLD signal 112 may correspond to any other active noise cancellation signal. Any of the signals described herein that are driven into a person may be low intensity so as to be safely driven into a person. Accordingly, any such signal is generally not noticeable to a person and it complies with the applicable health and safety guidelines (e.g., IEC 62368-1 ES1, IEC 60601, and the like). Driving electrode 116 may exhibit feedback impedance 114 that may vary based on body 102. For example, low moisture content (e.g., dry skin) may increase feedback impedance 114 whereas high moisture content (e.g., moist skin), may reduce feedback impedance 114. In some examples, it may be desirable to hold feedback impedance 114 to a minimum value.

RLD circuit 115 may also include tuning circuit 110 and/or control circuit 120 that may dynamically tune RLD signal 112 to more effectively cancel or counter noise in biosignal 138 from interference 104. For example, tuning circuit 110 and/or control circuit 120 may receive biosignal 138 (which may be amplified) from input amplifier 132. Based on characteristics of biosignal 138, control circuit 120 may control tuning circuit 110 to tune RLD signal 112. In some examples, control circuit 120 may include a microprocessor, controller, or other processor which may be separate from and/or integrated with a processor of AFE 130. Tuning RLD signal 112 may include, for example, adjusting a gain, frequency shaping for changing an amplitude of RLD signal 112 for various frequency components, etc. As will be described further below with respect to FIGS. 2A-2C, RLD circuit 115 (e.g., tuning circuit 110 and control circuit 120) may be implemented in an analog domain, digital domain, or mixed domain system.

FIGS. 2A-2C respectively illustrate an RLD circuit 215A, an RLD circuit 215B, and an RLD circuit 215C, each of which may correspond to RLD circuit 115. FIG. 2A illustrates RLD circuit 215A that may include a tuning circuit 210A which may correspond to tuning circuit 110, and a control circuit 220A which may correspond to control circuit 120. RLD circuit 215A may correspond to an analog domain implementation wherein tuning circuit 210A may include a voltage-controlled filter (VCF) 242 and a voltage-controlled amplifier (VCA) 244. Control circuit 220A may provide a control voltage to VCF 242 and/or VCA 244 for tuning, for instance by filtering out interference and increasing power, an RLD signal (e.g., RLD signal 112). Although a larger gain may be desirable, in some examples, to overcome a gain limitation, a voltage rail of RLD circuit 215A may be greater than a voltage rail of a corresponding analog front end (e.g., AFE 130). In such examples, RLD circuit 115 may operate using a higher voltage than that of AFE 130.

FIG. 2B illustrates RLD circuit 215B that may include a tuning circuit 210B which may correspond to tuning circuit 110, and a control circuit 220B which may correspond to control circuit 120. RLD circuit 215B may correspond to a digital domain implementation wherein tuning circuit 210B may include an amplifier 254 and control circuit 220B may include a digital signal processor (DSP) 256. Amplifier 254 may correspond to a VCA or other amplifier for increasing a signal's power. DSP 256 may correspond to a DSP, microprocessor, controller, or other processor for analyzing signals. In some examples, DSP 256 may be coupled to a machine-learning system. DSP 256 may analyze a biosignal (e.g., biosignal 138) to disambiguate between noise (e.g., narrow band interference) and useful information in the biosignal. Based on the determined noise, RLD circuit 215B can drive and/or shape the RLD signal (e.g., RLD signal 112).

FIG. 2A illustrates RLD circuit 215C that may include a tuning circuit 210C which may correspond to tuning circuit 110, and a control circuit 220C which may correspond to control circuit 120. RLD circuit 215C may correspond to a mixed domain implementation wherein tuning circuit 210C may include a filter 262 and an amplifier 264 and control circuit 220B may include a DSP 266. Filter 262 may correspond to a VCF or other filter for filtering out certain frequencies from a signal (e.g., frequencies corresponding to interference). Amplifier 264 may correspond to a VCA or other amplifier for increasing a signal's power. DSP 266 may correspond to a DSP, processor, controller, microprocessor, machine-learning system, or any other circuit for analyzing/processing signals. RLD circuit 215C may dynamically tune an RLD signal in the analog and digital domains, as described herein.

FIG. 3 is a flow diagram of an exemplary method 300 for dynamic signal tuning for active noise cancellation. The steps shown in FIG. 3 may be performed by any suitable computer-executable code and/or computing system, including the system(s) illustrated in FIGS. 1, 2A-2C, 6A-6B, and/or 7A-7B. In one example, each of the steps shown in FIG. 3 may represent an algorithm whose structure includes and/or is represented by multiple sub-steps, examples of which will be provided in greater detail below.

As illustrated in FIG. 3, at step 302 one or more of the systems described herein may drive, using a biosensing device, a right-leg drive (RLD) signal. For example, RLD circuit 115 of biosensing device 105 may drive RLD signal 112.

In some embodiments, the term “RLD signal” may refer to a signal that is driven into a body to reduce interference when measuring biosignals from the body. In other examples, RLD signal may refer to an active noise cancellation signal for countering noise in a signal.

The systems described herein may perform step 302 in a variety of ways. In one example, the biosensing device may include a plurality of electrodes. For example, biosensing device 105 includes driving electrode 116 which may correspond to one or more electrodes for driving RLD signal 112. In some examples, driving electrode 116 may correspond to sensing electrode 136.

At step 304 one or more of the systems described herein may measure, using the biosensing device, a biosignal. For example, biosensing device 105 may measure biosignal 138 via sensing electrode 136.

The systems described herein may perform step 304 in a variety of ways. In one example, the biosensing device may include a plurality of electrodes such that measuring the biosignal includes measuring a plurality of biosignals using the plurality of electrodes. For example, sensing electrode 136 may correspond to multiple electrodes used to measure biosignal 138.

In some examples, the biosensing device may include an RLD sensing electrode such that measuring the biosignal includes measuring the biosignal in response to the RLD signal using the RLD sensing electrode. For example, sensing electrode 136 may correspond to or include a dedicated sense electrode. In some examples, the RLD sensing electrode may be shaped larger than other biosensing electrodes of sensing electrode 136.

At step 306 one or more of the systems described herein may determine, from the measured biosignal, noise within the biosignal. For example, RLD circuit 115 (e.g., control circuit 120 and/or tuning circuit 110) may determine noise within biosignal 138.

In some embodiments, the term “noise” may refer to any undesirable component of a signal. In some examples, noise may originate from correlated and/or uncorrelated external aggressors to a desired signal which may or may not be random in nature. Examples of noise include, without limitation, external sources such as PLI, a motor, other electrical devices, etc., as well as internal sources such as electrical properties and/or changes thereof, EMI from circuitry, device characteristics, etc.

The systems described herein may perform step 306 in a variety of ways. In one example, determining the noise further may include identifying narrow band interference. For example, control circuit 120 (e.g., DSP 256 and/or DSP 266) may analyze biosignal 138 to identify narrow band interference and/or other noise within biosignal 138.

In some examples, determining the noise may include processing the biosignal. For example, when sensing electrode 136 includes multiple electrodes measuring multiple biosignals 138, the average of the multiple biosignals 138 may be analyzed for noise.

At step 308 one or more of the systems described herein may tune, based on the noise, the RLD signal. For example, RLD circuit 115 (e.g., tuning circuit 110 and/or control circuit 120) may tune RLD signal 112 based on the noise.

In some embodiments, the term “tuning” may refer to making spectral changes in a signal. Examples of tuning a signal include, without limitation, filtering certain frequencies, changing amplitudes with respect to certain frequencies, changing an applied gain, synthesizing a signal to counter an unwanted signal, etc.

The systems described herein may perform step 308 in a variety of ways. In one example, wherein tuning the RLD signal may further include inverting the noise and feeding the inverted noise into the RLD signal. For example, RLD circuit 115 (e.g. RLD circuit 215A and/or RLD circuit 215C) may invert the noise and add the inverted noise to RLD signal 112.

In some examples, tuning the RLD signal may further include shaping the RLD signal based on the identified narrow band interference. For example RLD, circuit 115 (e.g., RLD circuit 215B and/or RLD circuit 215C) may shape RLD signal 112 by synthesizing a signal to counter the narrow band interference.

In some examples, tuning the RLD signal may further include frequency shaping the RLD signal to change an amplitude of the RLD signal for a frequency. For example RLD, circuit 115 (e.g., RLD circuit 215B and/or RLD circuit 215C) may shape RLD signal 112 by changing the amplitude of RLD signal 112 for a particular frequency. For example, to counter particular frequencies corresponding to noise within biosignal 138, RLD circuit 115 may accordingly change amplitudes of RLD signal 112 for the particular frequencies.

In some examples, tuning the RLD signal may further include tuning the RLD signal using a voltage rail that is larger than a voltage rail of an analog front end of the biosensing device. For example, RLD circuit 115 may operate with a voltage that is greater than a voltage at which AFE 130 operates. In some examples, operating RLD circuit 115 with a voltage rail that is independent from a voltage rail of AFE 130 may allow additional tuning of RLD signal 112. For instance, applying a gain factor of 200 on a 1v driven RLD signal may behave similarly to applying a gain factor of 100 on a 2v driven RLD signal. By having RLD circuit 115 operate at a different voltage rail than that of AFE 130, RLD circuit 115 may have a broader range of tuning than if RLD circuit 115 shared the same voltage rail as AFE 130.

In some examples, RLD circuit 115 may drive RLD signal 112 with different voltages which may reduce impedance (e.g., effective impedance 134 and/or feedback impedance 114). In some examples, reducing the impedance may reduce or cancel noise.

In some examples, RLD circuit 115 may continuously and/or dynamically tune RLD signal 112 in response to changes in the noise in biosignal 138. For example, changes with biosensing device 105 with respect to body 102 (e.g., displacement, electrode liftoff, etc.), changes to an environment and/or condition of body 102 (moving to different locations or otherwise experiencing changes to interference 104, changes in a skin of body 102), and/or previously tuned RLD signal 112 may cause changes to the noise in biosignal 138. RLD circuit 115 may accordingly re-tune RLD signal 112 in response to such changes.

In reference to the systems and methods described herein, active noise cancellation via right-leg drive may be more effective with maximum gain and minimum contact impedance. However, a static implementation may suffer from instability. As such, a dynamic control of loop gain may allow for greater efficacy while preventing instability.

Dynamic tuning of the RLD block may prevent oscillation in the event of electrode liftoff, increase phase margin, reduce noise, and increase the signal-to-noise ratio (SNR) of a biosensing application. The RLD block may also utilize frequency shaping techniques to change the amplitude of the RLD feedback for various frequency components which may reduce or minimize specific noise sources or maximize EMG signal in particular bands. The dynamically tunable RLD circuit may be implemented in the digital domain, analog domain, or be a mixed domain system.

An RLD system gain may be maintained as high as possible, bringing the common mode of the person close to system ground to make the elimination of common-mode noise possible regardless of sensing architecture. Additionally, voltage rails beyond the supply may improve performance.

In one example, by considering an RLD signal going into a body as a transfer function, the systems and methods described herein may act to dynamically tune this transfer function. In some examples, an RLD circuit may have a fixed feedback loop. The systems and methods described herein may provide a second feedback loop for tuning the fixed feedback loop.

As detailed above, the RLD system provided herein may provide certain advantages, such as advantageously maximizing loop gain (e.g., for cancelling out unwanted frequencies) while maintaining stability (e.g., without signal oscillating). As described above, an active element (e.g., digitally and/or via filter circuits) in signal feedback (which may be digital and/or analog) may allow increasing gain closer to a gain limit causing instability (e.g., by detecting oscillation and accordingly shaping a feedback to reduce the detected oscillation).

Moreover, the systems and methods described herein may allow multiple RLD signals/systems for a body, further allowing independent tuning of each RLD signal. In some examples, the multiple RLD systems (e.g., multiple iterations of RLD circuit 115, which may further correspond to one or more of RLD circuit 215A, RLD circuit 215B, and/or RLD circuit 215C) may be parallel, such as serving as a redundant system as well as existing in multiple places in the same system (e.g., in biosensing device 105). In some examples, the multiple RLD systems may be each tuned for addressing different interference frequencies, such as addressing a particular interference frequency (e.g., 60 Hz for power line interference) when closer to a source of such interference, and/or addressing another interference frequency when closer to a corresponding source of interference (e.g., an electric vehicle and/or other electric appliance).

In some examples, the systems and methods described herein allow dynamic tuning in response to changing conditions of electrodes, such as distances from interference sources, physiological and/or environmental changes (e.g., sweating, changes in humidity), etc.

The systems and methods described herein may improve a system settling time as well as reduce noise. For instance, higher gain may increase an effective area of an electrode (and/or reduces impedance of the electrode interface). Reducing reference impedance may reduce system settling time such that a higher gain may allow reducing interference more quickly than with a lesser gain. The higher gain may also reduce an impact of the impedance exhibited by the skin-electrode interface.

### Example Embodiments

Example 1: A method comprising: driving, using a biosensing device, a right-leg drive (RLD) signal; measuring, using the biosensing device, a biosignal; determining, from the measured biosignal, noise within the biosignal; and tuning, based on the noise, the RLD signal.
Example 2: The method of Example 1, wherein tuning the RLD signal further comprises: inverting the noise; and feeding the inverted noise into the RLD signal.
Example 3: The method of Example 1 or 2, wherein: determining the noise further comprises identifying narrow band interference; and tuning the RLD signal further comprises shaping the RLD signal based on the identified narrow band interference.
Example 4: The method of Example 1, 2, or 3, wherein tuning the RLD signal further comprises tuning the RLD signal using a voltage rail that is larger than a voltage rail of an analog front end of the biosensing device.
Example 5: The method of any of Examples 1-4, wherein: the biosensing device comprises a plurality of electrodes; measuring the biosignal comprises measuring a plurality of biosignals using the plurality of electrodes; and determining the noise is further based on an average of the plurality of biosignals.
Example 6: The method of any of Examples 1-5, wherein: the biosensing device comprises an RLD sensing electrode; and measuring the biosignal comprises measuring the biosignal in response to the RLD signal using the RLD sensing electrode.
Example 7: The method of Example 6, wherein the RLD sensing electrode is larger than a biosensing electrode of the biosensing device.
Example 8: The method of any of Examples 1-7, wherein tuning the RLD signal further comprises frequency shaping the RLD signal to change an amplitude of the RLD signal for a frequency.
Example 9: A biosensing device comprising: at least one electrode for biosignal measurement; a right-leg drive (RLD) circuit comprising a tuning circuit and a control circuit; and at least one physical processor configured to: drive, using the RLD circuit, an RLD signal; measure, using the at least one electrode, a biosignal; determine, from the measured biosignal, noise within the biosignal; and tune, based on the noise using the tuning circuit, the RLD signal.
Example 10: The biosensing device of Example 9, wherein tuning the RLD signal further comprises: inverting the noise; and feeding the inverted noise into the RLD signal.
Example 11: The biosensing device of Example 9 or 10, wherein: determining the noise further comprises identifying narrow band interference; and tuning the RLD signal further comprises shaping the RLD signal based on the identified narrow band interference.
Example 12: The biosensing device of Example 9, 10, or 11, further comprising: an analog front end for processing the biosignal and having a first voltage rail; wherein the RLD circuit has a second voltage rail at a greater voltage than a voltage of the first voltage rail.
Example 13: The biosensing device of any of Examples 9-12, further comprising a plurality of electrodes, wherein: measuring the biosignal comprises measuring a plurality of biosignals using the plurality of electrodes; and determining the noise is further based on an average of the plurality of biosignals.
Example 14: The biosensing device of any of Examples 9-13, wherein the at least one electrode comprises an RLD sensing electrode, wherein measuring the biosignal comprises measuring the biosignal in response to the RLD signal using the RLD sensing electrode.
Example 15: The biosensing device of Example 14, wherein the at least one electrode comprises a biosensing electrode and the RLD sensing electrode is larger than the biosensing electrode.
Example 16: The biosensing device of any of Examples 9-15, wherein tuning the RLD signal further comprises frequency shaping the RLD signal to change an amplitude of the RLD signal for a frequency.
Example 17: A system comprising: at least one physical processor; physical memory comprising computer-executable instructions; and a biosensing device comprising: at least one electrode for biosignal measurement; and a right-leg drive (RLD) circuit comprising a tuning circuit and a control circuit; wherein the at least one physical processor is configured to: drive, using the RLD circuit, an RLD signal; measure, using the at least one electrode, a biosignal; determine, from the measured biosignal, noise within the biosignal; and tune, based on the noise using the tuning circuit, the RLD signal.
Example 18: The system of Example 17, wherein tuning the RLD signal further comprises at least one of: feeding an inversion of the noise into the RLD signal; shaping the RLD signal based on identifying narrow band interference within the biosignal; or frequency shaping the RLD signal to change an amplitude of the RLD signal fora frequency.
Example 19: The system of Example 17 or 18, wherein: the biosensing device comprises an analog front end for processing the biosignal and having a first voltage rail; and the RLD circuit has a second voltage rail at a greater voltage than a voltage of the first voltage rail.
Example 20: The system of Example 17, 18, or 19, wherein: the at least one electrode comprises a biosensing electrode and an RLD sensing electrode larger than the biosensing electrode; and measuring the biosignal comprises measuring the biosignal in response to the RLD signal using the RLD sensing electrode.

Embodiments of the present disclosure may include or be implemented in conjunction with various types of artificial-reality systems. Artificial reality is a form of reality that has been adjusted in some manner before presentation to a user, which may include, for example, a virtual reality, an augmented reality, a mixed reality, a hybrid reality, or some combination and/or derivative thereof. Artificial-reality content may include completely computer-generated content or computer-generated content combined with captured (e.g., real-world) content. The artificial-reality content may include video, audio, haptic feedback, or some combination thereof, any of which may be presented in a single channel or in multiple channels (such as stereo video that produces a three-dimensional (3D) effect to the viewer). Additionally, in some embodiments, artificial reality may also be associated with applications, products, accessories, services, or some combination thereof, that are used to, for example, create content in an artificial reality and/or are otherwise used in (e.g., to perform activities in) an artificial reality.

Artificial-reality systems may be implemented in a variety of different form factors and configurations. Some artificial-reality systems may be designed to work without near-eye displays (NEDs). Other artificial-reality systems may include an NED that also provides visibility into the real world (such as, e.g., augmented-reality system 400 in FIG. 4) or that visually immerses a user in an artificial reality (such as, e.g., virtual-reality system 500 in FIG. 5). While some artificial-reality devices may be self-contained systems, other artificial-reality devices may communicate and/or coordinate with external devices to provide an artificial-reality experience to a user. Examples of such external devices include handheld controllers, mobile devices, desktop computers, devices worn by a user, devices worn by one or more other users, and/or any other suitable external system.

Turning to FIG. 4, augmented-reality system 400 may include an eyewear device 402 with a frame 410 configured to hold a left display device 415(A) and a right display device 415(B) in front of a user's eyes. Display devices 415(A) and 415(B) may act together or independently to present an image or series of images to a user. While augmented-reality system 400 includes two displays, embodiments of this disclosure may be implemented in augmented-reality systems with a single NED or more than two NEDs.

In some embodiments, augmented-reality system 400 may include one or more sensors, such as sensor 440. Sensor 440 may generate measurement signals in response to motion of augmented-reality system 400 and may be located on substantially any portion of frame 410. Sensor 440 may represent one or more of a variety of different sensing mechanisms, such as a position sensor, an inertial measurement unit (IMU), a depth camera assembly, a structured light emitter and/or detector, or any combination thereof. In some embodiments, augmented-reality system 400 may or may not include sensor 440 or may include more than one sensor. In embodiments in which sensor 440 includes an IMU, the IMU may generate calibration data based on measurement signals from sensor 440. Examples of sensor 440 may include, without limitation, accelerometers, gyroscopes, magnetometers, other suitable types of sensors that detect motion, sensors used for error correction of the IMU, or some combination thereof.

In some examples, augmented-reality system 400 may also include a microphone array with a plurality of acoustic transducers 420(A)-420(J), referred to collectively as acoustic transducers 420. Acoustic transducers 420 may represent transducers that detect air pressure variations induced by sound waves. Each acoustic transducer 420 may be configured to detect sound and convert the detected sound into an electronic format (e.g., an analog or digital format). The microphone array in FIG. 4 may include, for example, ten acoustic transducers: 420(A) and 420(B), which may be designed to be placed inside a corresponding ear of the user, acoustic transducers 420(C), 420(D), 420(E), 420(F), 420(G), and 420(H), which may be positioned at various locations on frame 410, and/or acoustic transducers 420(1) and 420(J), which may be positioned on a corresponding neckband 405.

In some embodiments, one or more of acoustic transducers 420(A)-(J) may be used as output transducers (e.g., speakers). For example, acoustic transducers 420(A) and/or 420(B) may be earbuds or any other suitable type of headphone or speaker.

The configuration of acoustic transducers 420 of the microphone array may vary. While augmented-reality system 400 is shown in FIG. 4 as having ten acoustic transducers 420, the number of acoustic transducers 420 may be greater or less than ten. In some embodiments, using higher numbers of acoustic transducers 420 may increase the amount of audio information collected and/or the sensitivity and accuracy of the audio information. In contrast, using a lower number of acoustic transducers 420 may decrease the computing power required by an associated controller 450 to process the collected audio information. In addition, the position of each acoustic transducer 420 of the microphone array may vary. For example, the position of an acoustic transducer 420 may include a defined position on the user, a defined coordinate on frame 410, an orientation associated with each acoustic transducer 420, or some combination thereof.

Acoustic transducers 420(A) and 420(B) may be positioned on different parts of the user's ear, such as behind the pinna, behind the tragus, and/or within the auricle or fossa. Or, there may be additional acoustic transducers 420 on or surrounding the ear in addition to acoustic transducers 420 inside the ear canal. Having an acoustic transducer 420 positioned next to an ear canal of a user may enable the microphone array to collect information on how sounds arrive at the ear canal. By positioning at least two of acoustic transducers 420 on either side of a user's head (e.g., as binaural microphones), augmented-reality system 400 may simulate binaural hearing and capture a 3D stereo sound field around about a user's head. In some embodiments, acoustic transducers 420(A) and 420(B) may be connected to augmented-reality system 400 via a wired connection 430, and in other embodiments acoustic transducers 420(A) and 420(B) may be connected to augmented-reality system 400 via a wireless connection (e.g., a BLUETOOTH connection). In still other embodiments, acoustic transducers 420(A) and 420(B) may not be used at all in conjunction with augmented-reality system 400.

Acoustic transducers 420 on frame 410 may be positioned in a variety of different ways, including along the length of the temples, across the bridge, above or below display devices 415(A) and 415(B), or some combination thereof. Acoustic transducers 420 may also be oriented such that the microphone array is able to detect sounds in a wide range of directions surrounding the user wearing the augmented-reality system 400. In some embodiments, an optimization process may be performed during manufacturing of augmented-reality system 400 to determine relative positioning of each acoustic transducer 420 in the microphone array.

In some examples, augmented-reality system 400 may include or be connected to an external device (e.g., a paired device), such as neckband 405. Neckband 405 generally represents any type or form of paired device. Thus, the following discussion of neckband 405 may also apply to various other paired devices, such as charging cases, smart watches, smart phones, wrist bands, other wearable devices, hand-held controllers, tablet computers, laptop computers, other external compute devices, etc.

As shown, neckband 405 may be coupled to eyewear device 402 via one or more connectors. The connectors may be wired or wireless and may include electrical and/or non-electrical (e.g., structural) components. In some cases, eyewear device 402 and neckband 405 may operate independently without any wired or wireless connection between them. While FIG. 4 illustrates the components of eyewear device 402 and neckband 405 in example locations on eyewear device 402 and neckband 405, the components may be located elsewhere and/or distributed differently on eyewear device 402 and/or neckband 405. In some embodiments, the components of eyewear device 402 and neckband 405 may be located on one or more additional peripheral devices paired with eyewear device 402, neckband 405, or some combination thereof.

Pairing external devices, such as neckband 405, with augmented-reality eyewear devices may enable the eyewear devices to achieve the form factor of a pair of glasses while still providing sufficient battery and computation power for expanded capabilities. Some or all of the battery power, computational resources, and/or additional features of augmented-reality system 400 may be provided by a paired device or shared between a paired device and an eyewear device, thus reducing the weight, heat profile, and form factor of the eyewear device overall while still retaining desired functionality. For example, neckband 405 may allow components that would otherwise be included on an eyewear device to be included in neckband 405 since users may tolerate a heavier weight load on their shoulders than they would tolerate on their heads. Neckband 405 may also have a larger surface area over which to diffuse and disperse heat to the ambient environment. Thus, neckband 405 may allow for greater battery and computation capacity than might otherwise have been possible on a stand-alone eyewear device. Since weight carried in neckband 405 may be less invasive to a user than weight carried in eyewear device 402, a user may tolerate wearing a lighter eyewear device and carrying or wearing the paired device for greater lengths of time than a user would tolerate wearing a heavy standalone eyewear device, thereby enabling users to more fully incorporate artificial-reality environments into their day-to-day activities.

Neckband 405 may be communicatively coupled with eyewear device 402 and/or to other devices. These other devices may provide certain functions (e.g., tracking, localizing, depth mapping, processing, storage, etc.) to augmented-reality system 400. In the embodiment of FIG. 4, neckband 405 may include two acoustic transducers (e.g., 420(1) and 420(J)) that are part of the microphone array (or potentially form their own microphone subarray). Neckband 405 may also include a controller 425 and a power source 435.

Acoustic transducers 420(1) and 420(J) of neckband 405 may be configured to detect sound and convert the detected sound into an electronic format (analog or digital). In the embodiment of FIG. 4, acoustic transducers 420(1) and 420(J) may be positioned on neckband 405, thereby increasing the distance between the neckband acoustic transducers 420(1) and 420(J) and other acoustic transducers 420 positioned on eyewear device 402. In some cases, increasing the distance between acoustic transducers 420 of the microphone array may improve the accuracy of beamforming performed via the microphone array. For example, if a sound is detected by acoustic transducers 420(C) and 420(D) and the distance between acoustic transducers 420(C) and 420(D) is greater than, e.g., the distance between acoustic transducers 420(D) and 420(E), the determined source location of the detected sound may be more accurate than if the sound had been detected by acoustic transducers 420(D) and 420(E).

Controller 425 of neckband 405 may process information generated by the sensors on neckband 405 and/or augmented-reality system 400. For example, controller 425 may process information from the microphone array that describes sounds detected by the microphone array. For each detected sound, controller 425 may perform a direction-of-arrival (DOA) estimation to estimate a direction from which the detected sound arrived at the microphone array. As the microphone array detects sounds, controller 425 may populate an audio data set with the information. In embodiments in which augmented-reality system 400 includes an inertial measurement unit, controller 425 may compute all inertial and spatial calculations from the IMU located on eyewear device 402. A connector may convey information between augmented-reality system 400 and neckband 405 and between augmented-reality system 400 and controller 425. The information may be in the form of optical data, electrical data, wireless data, or any other transmittable data form. Moving the processing of information generated by augmented-reality system 400 to neckband 405 may reduce weight and heat in eyewear device 402, making it more comfortable to the user.

Power source 435 in neckband 405 may provide power to eyewear device 402 and/or to neckband 405. Power source 435 may include, without limitation, lithium ion batteries, lithium-polymer batteries, primary lithium batteries, alkaline batteries, or any other form of power storage. In some cases, power source 435 may be a wired power source. Including power source 435 on neckband 405 instead of on eyewear device 402 may help better distribute the weight and heat generated by power source 435.

As noted, some artificial-reality systems may, instead of blending an artificial reality with actual reality, substantially replace one or more of a user's sensory perceptions of the real world with a virtual experience. One example of this type of system is a head-worn display system, such as virtual-reality system 500 in FIG. 5, that mostly or completely covers a user's field of view. Virtual-reality system 500 may include a front rigid body 502 and a band 504 shaped to fit around a user's head. Virtual-reality system 500 may also include output audio transducers 506(A) and 506(B). Furthermore, while not shown in FIG. 5, front rigid body 502 may include one or more electronic elements, including one or more electronic displays, one or more inertial measurement units (IMUS), one or more tracking emitters or detectors, and/or any other suitable device or system for creating an artificial-reality experience.

Artificial-reality systems may include a variety of types of visual feedback mechanisms. For example, display devices in augmented-reality system 400 and/or virtual-reality system 500 may include one or more liquid crystal displays (LCDs), light emitting diode (LED) displays, microLED displays, organic LED (OLED) displays, digital light project (DLP) micro-displays, liquid crystal on silicon (LCoS) micro-displays, and/or any other suitable type of display screen. These artificial-reality systems may include a single display screen for both eyes or may provide a display screen for each eye, which may allow for additional flexibility for varifocal adjustments or for correcting a user's refractive error. Some of these artificial-reality systems may also include optical subsystems having one or more lenses (e.g., concave or convex lenses, Fresnel lenses, adjustable liquid lenses, etc.) through which a user may view a display screen. These optical subsystems may serve a variety of purposes, including to collimate (e.g., make an object appear at a greater distance than its physical distance), to magnify (e.g., make an object appear larger than its actual size), and/or to relay (to, e.g., the viewer's eyes) light. These optical subsystems may be used in a non-pupil-forming architecture (such as a single lens configuration that directly collimates light but results in so-called pincushion distortion) and/or a pupil-forming architecture (such as a multi-lens configuration that produces so-called barrel distortion to nullify pincushion distortion).

In addition to or instead of using display screens, some of the artificial-reality systems described herein may include one or more projection systems. For example, display devices in augmented-reality system 400 and/or virtual-reality system 500 may include microLED projectors that project light (using, e.g., a waveguide) into display devices, such as clear combiner lenses that allow ambient light to pass through. The display devices may refract the projected light toward a user's pupil and may enable a user to simultaneously view both artificial-reality content and the real world. The display devices may accomplish this using any of a variety of different optical components, including waveguide components (e.g., holographic, planar, diffractive, polarized, and/or reflective waveguide elements), light-manipulation surfaces and elements (such as diffractive, reflective, and refractive elements and gratings), coupling elements, etc. Artificial-reality systems may also be configured with any other suitable type or form of image projection system, such as retinal projectors used in virtual retina displays.

The artificial-reality systems described herein may also include various types of computer vision components and subsystems. For example, augmented-reality system 400 and/or virtual-reality system 500 may include one or more optical sensors, such as two-dimensional (2D) or 3D cameras, structured light transmitters and detectors, time-of-flight depth sensors, single-beam or sweeping laser rangefinders, 3D LiDAR sensors, and/or any other suitable type or form of optical sensor. An artificial-reality system may process data from one or more of these sensors to identify a location of a user, to map the real world, to provide a user with context about real-world surroundings, and/or to perform a variety of other functions.

The artificial-reality systems described herein may also include one or more input and/or output audio transducers. Output audio transducers may include voice coil speakers, ribbon speakers, electrostatic speakers, piezoelectric speakers, bone conduction transducers, cartilage conduction transducers, tragus-vibration transducers, and/or any other suitable type or form of audio transducer. Similarly, input audio transducers may include condenser microphones, dynamic microphones, ribbon microphones, and/or any other type or form of input transducer. In some embodiments, a single transducer may be used for both audio input and audio output.

In some embodiments, the artificial-reality systems described herein may also include tactile (i.e., haptic) feedback systems, which may be incorporated into headwear, gloves, body suits, handheld controllers, environmental devices (e.g., chairs, floormats, etc.), and/or any other type of device or system. Haptic feedback systems may provide various types of cutaneous feedback, including vibration, force, traction, texture, and/or temperature. Haptic feedback systems may also provide various types of kinesthetic feedback, such as motion and compliance. Haptic feedback may be implemented using motors, piezoelectric actuators, fluidic systems, and/or a variety of other types of feedback mechanisms. Haptic feedback systems may be implemented independent of other artificial-reality devices, within other artificial-reality devices, and/or in conjunction with other artificial-reality devices.

By providing haptic sensations, audible content, and/or visual content, artificial-reality systems may create an entire virtual experience or enhance a user's real-world experience in a variety of contexts and environments. For instance, artificial-reality systems may assist or extend a user's perception, memory, or cognition within a particular environment. Some systems may enhance a user's interactions with other people in the real world or may enable more immersive interactions with other people in a virtual world. Artificial-reality systems may also be used for educational purposes (e.g., for teaching or training in schools, hospitals, government organizations, military organizations, business enterprises, etc.), entertainment purposes (e.g., for playing video games, listening to music, watching video content, etc.), and/or for accessibility purposes (e.g., as hearing aids, visual aids, etc.). The embodiments disclosed herein may enable or enhance a user's artificial-reality experience in one or more of these contexts and environments and/or in other contexts and environments.

FIG. 6A illustrates an exemplary human-machine interface (also referred to herein as an EMG control interface) configured to be worn around a user's lower arm or wrist as a wearable system 600. In this example, wearable system 600 may include sixteen neuromuscular sensors 610 (e.g., EMG sensors) arranged circumferentially around an elastic band 620 with an interior surface 630 configured to contact a user's skin. However, any suitable number of neuromuscular sensors may be used. The number and arrangement of neuromuscular sensors may depend on the particular application for which the wearable device is used. For example, a wearable armband or wristband can be used to generate control information for controlling an augmented reality system, a robot, controlling a vehicle, scrolling through text, controlling a virtual avatar, or any other suitable control task. As shown, the sensors may be coupled together using flexible electronics incorporated into the wireless device. FIG. 6B illustrates a cross-sectional view through one of the sensors of the wearable device shown in FIG. 6A. In some embodiments, the output of one or more of the sensing components can be optionally processed using hardware signal processing circuitry (e.g., to perform amplification, filtering, and/or rectification). In other embodiments, at least some signal processing of the output of the sensing components can be performed in software. Thus, signal processing of signals sampled by the sensors can be performed in hardware, software, or by any suitable combination of hardware and software, as aspects of the technology described herein are not limited in this respect. A non-limiting example of a signal processing chain used to process recorded data from sensors 610 is discussed in more detail below with reference to FIGS. 7A and 7B.

FIGS. 7A and 7B illustrate an exemplary schematic diagram with internal components of a wearable system with EMG sensors. As shown, the wearable system may include a wearable portion 710 (FIG. 7A) and a dongle portion 720 (FIG. 7B) in communication with the wearable portion 710 (e.g., via BLUETOOTH or another suitable wireless communication technology). As shown in FIG. 7A, the wearable portion 710 may include skin contact electrodes 711, examples of which are described in connection with FIGS. 6A and 6B. The output of the skin contact electrodes 711 may be provided to analog front end 730, which may be configured to perform analog processing (e.g., amplification, noise reduction, filtering, etc.) on the recorded signals. The processed analog signals may then be provided to analog-to-digital converter 732, which may convert the analog signals to digital signals that can be processed by one or more computer processors. An example of a computer processor that may be used in accordance with some embodiments is microcontroller (MCU) 734, illustrated in FIG. 7A. As shown, MCU 734 may also include inputs from other sensors (e.g., IMU sensor 740), and power and battery module 742. The output of the processing performed by MCU 734 may be provided to antenna 750 for transmission to dongle portion 720 shown in FIG. 7B.

Dongle portion 720 may include antenna 752, which may be configured to communicate with antenna 750 included as part of wearable portion 710. Communication between antennas 750 and 752 may occur using any suitable wireless technology and protocol, non-limiting examples of which include radiofrequency signaling and BLUETOOTH. As shown, the signals received by antenna 752 of dongle portion 720 may be provided to a host computer for further processing, display, and/or for effecting control of a particular physical or virtual object or objects.

Although the examples provided with reference to FIGS. 6A-6B and FIGS. 7A-7B are discussed in the context of interfaces with EMG sensors, the techniques described herein for reducing electromagnetic interference can also be implemented in wearable interfaces with other types of sensors including, but not limited to, mechanomyography (MMG) sensors, sonomyography (SMG) sensors, and electrical impedance tomography (EIT) sensors. The techniques described herein for reducing electromagnetic interference can also be implemented in wearable interfaces that communicate with computer hosts through wires and cables (e.g., USB cables, optical fiber cables, etc.).

In some examples, the term “memory device” generally refers to any type or form of volatile or non-volatile storage device or medium capable of storing data and/or computer-readable instructions. In one example, a memory device may store, load, and/or maintain one or more of the modules described herein. Examples of memory devices include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In some examples, the term “physical processor” generally refers to any type or form of hardware-implemented processing unit capable of interpreting and/or executing computer-readable instructions. In one example, a physical processor may access and/or modify one or more modules stored in the above-described memory device. Examples of physical processors include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

In some embodiments, the term “computer-readable medium” generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A method comprising:
driving, using a biosensing device, a right-leg drive (RLD) signal;
measuring, using the biosensing device, a biosignal;
determining, from the measured biosignal, noise within the biosignal; and
tuning, based on the noise, the RLD signal.

2. The method of claim 1, wherein tuning the RLD signal further comprises:
inverting the noise; and
feeding the inverted noise into the RLD signal.

3. The method of claim 1, wherein:
determining the noise further comprises identifying narrow band interference; and
tuning the RLD signal further comprises shaping the RLD signal based on the identified narrow band interference.

4. The method of claim 1, wherein tuning the RLD signal further comprises tuning the RLD signal using a voltage rail that is larger than a voltage rail of an analog front end of the biosensing device.

5. The method of claim 1, wherein:
the biosensing device comprises a plurality of electrodes;
measuring the biosignal comprises measuring a plurality of biosignals using the plurality of electrodes; and
determining the noise is further based on an average of the plurality of biosignals.

6. The method of claim 1, wherein:
the biosensing device comprises an RLD sensing electrode; and
measuring the biosignal comprises measuring the biosignal in response to the RLD signal using the RLD sensing electrode.

7. The method of claim 6, wherein the RLD sensing electrode is larger than a biosensing electrode of the biosensing device.

8. The method of claim 1, wherein tuning the RLD signal further comprises frequency shaping the RLD signal to change an amplitude of the RLD signal for a frequency.

9. A biosensing device comprising:
at least one electrode for biosignal measurement;
a right-leg drive (RLD) circuit comprising a tuning circuit and a control circuit; and
at least one physical processor configured to:
drive, using the RLD circuit, an RLD signal;
measure, using the at least one electrode, a biosignal;
determine, from the measured biosignal, noise within the biosignal; and
tune, based on the noise using the tuning circuit, the RLD signal.

10. The biosensing device of claim 9, wherein tuning the RLD signal further comprises:
inverting the noise; and
feeding the inverted noise into the RLD signal.

11. The biosensing device of claim 9, wherein:
determining the noise further comprises identifying narrow band interference; and
tuning the RLD signal further comprises shaping the RLD signal based on the identified narrow band interference.

12. The biosensing device of claim 9, further comprising:
an analog front end for processing the biosignal and having a first voltage rail;
wherein the RLD circuit has a second voltage rail at a greater voltage than a voltage of the first voltage rail.

13. The biosensing device of claim 9, further comprising a plurality of electrodes, wherein:
measuring the biosignal comprises measuring a plurality of biosignals using the plurality of electrodes; and
determining the noise is further based on an average of the plurality of biosignals.

14. The biosensing device of claim 9, wherein the at least one electrode comprises an RLD sensing electrode, wherein measuring the biosignal comprises measuring the biosignal in response to the RLD signal using the RLD sensing electrode.

15. The biosensing device of claim 14, wherein the at least one electrode comprises a biosensing electrode and the RLD sensing electrode is larger than the biosensing electrode.

16. The biosensing device of claim 9, wherein tuning the RLD signal further comprises frequency shaping the RLD signal to change an amplitude of the RLD signal for a frequency.

17. A system comprising:
at least one physical processor;
physical memory comprising computer-executable instructions; and
a biosensing device comprising:
at least one electrode for biosignal measurement; and
a right-leg drive (RLD) circuit comprising a tuning circuit and a control circuit;
wherein the at least one physical processor is configured to:
drive, using the RLD circuit, an RLD signal;
measure, using the at least one electrode, a biosignal;
determine, from the measured biosignal, noise within the biosignal; and
tune, based on the noise using the tuning circuit, the RLD signal.

18. The system of claim 17, wherein tuning the RLD signal further comprises at least one of:
feeding an inversion of the noise into the RLD signal;
shaping the RLD signal based on identifying narrow band interference within the biosignal; or
frequency shaping the RLD signal to change an amplitude of the RLD signal for a frequency.

19. The system of claim 17, wherein:
the biosensing device comprises an analog front end for processing the biosignal and having a first voltage rail; and
the RLD circuit has a second voltage rail at a greater voltage than a voltage of the first voltage rail.

20. The system of claim 17, wherein:
the at least one electrode comprises a biosensing electrode and an RLD sensing electrode larger than the biosensing electrode; and
measuring the biosignal comprises measuring the biosignal in response to the RLD signal using the RLD sensing electrode.
